(19) United States
(12) Patent Application Publication
(10) Pub. No.: US 2018/0110952 A1
(43) Pub. Date: Apr. 26, 2018

(54) METHODS AND APPARATUS FOR REDUCING ANXIETY IN A PATIENT

(71) Applicant: Charles H. Bowen, O'Fallon, MO (US)

(72) Inventor: Charles H. Bowen, O'Fallon, MO (US)

(21) Appl. No.: 15/789,272

(22) Filed: Oct. 20, 2017

Related U.S. Application Data
(60) Provisional application No. 62/411,291, filed on Oct. 21, 2016.

(51) Int. Cl. A61M 16/06; A61K 31/075

(52) U.S. Cl. CPC A61M 16/0605; A61M 16/20; A61K 31/075

(57) ABSTRACT

Apparatus and methods for inhibiting the odor of an anesthesia mask, and thereby reducing patient anxiety, generally comprising the use of a towelette treated with a pleasant odorant or flavorant to wipe down a piece of medical equipment, generally a piece of non-metallic or plastic equipment, such as a breathing mask, prior to it being positioned on a patient, and in a location on the patient where the odorant or flavorant can be smelled or tasted.

### CROSS-REFERENCE TO RELATED APPLICATIONS

This Application claims benefit of U.S. Provisional Patent Application No. 62/411,291 filed Oct. 21, 2016, the entire disclosure of which is incorporated herein by reference.

### BACKGROUND OF THE INVENTION

### Field of the Invention

This disclosure relates to reducing anxiety in patients during medical procedures, and more particularly, to applying a scented towelette to a breathing mask to obscure the unpleasant odor of such masks.

### Description of the Related Art

Anesthesia is temporary, medically-induced state of unconsciousness, or reduced consciousness, in a patient. Anesthesia is typically used in order to painlessly perform medical procedures that would otherwise produce severe pain, or to sedate patients who might otherwise be too anxious or uncooperative to perform the medical procedure effectively. Anesthesia is typically characterized by analgesia, paralysis, and unconsciousness. General anesthesia suppresses the central nervous system, producing unconsciousness and usually a complete absence of sensation in the patient. Local anesthesia, by contrast, blocks the transmission of nerve impulses between the central nervous system and another part of the body, causing loss of sensation in the isolated part, but without suppressing the central nervous system. Finally, sedation is a suppression of the central nervous system to a lesser degree than a general anesthesia, resulting in a reduced level of consciousness, reducing anxiety and memory of the event, but without full loss of consciousness.

Historically, anesthetic compounds achieved the goals of paralysis and unconsciousness, but often did not sufficient inhibit the body's stress response to the medical procedure being performed, resulting in blood pressure and pulse spikes that could prove fatal. In modern practice, it is common to use an inhaled anesthetic, which operates on the spinal cord, in conjunction with an injected anesthetic introduced intravenously, which operates directly on the brain. The combination of anesthetics is typically more effective than any one compound. For example, a patient undergoing general anesthesia may be started off with an injection of propofol to start the anesthetic, then fentanyl to suppress the stress response and midazolam to facilitate amnesia. The patient may also be fitted with a breathing mask and supplied thereby with a sevoflurane inhalant during the procedure to maintain the effects.

A number of inhaled anesthetics are known, most of them being fluorinated ethers such as sevoflurane, isoflurane, enflurane, and desflurane. Other known, and less volatile, anesthetic inhalants include nitrous oxide and xenon. The commonly used anesthetic inhalants are largely odorless, and not usually delivered to the patient until after the patient loses consciousness from the injected anesthetics. However, a breathing mask is often fitted to the patient before he or she loses consciousness to supply oxygen and ensure that the patient remains safe during the process.

Many patients find the odor of the breathing mask, colloquially referred as the “new plastic” smell, highly unpleasant. This is particularly true with children, who are often more sensitive to smells and tastes than adults. This unfamiliar odor is sometimes mistaken for the smell of the anesthetic inhalant itself Many patients are anxious about anesthesia, particularly general anesthesia. The clinical odor of the mask, combined with the patient's concern over the anesthesia and procedure to be performed, may cause increased anxiety in the patient. This is particularly true with children, who are often more attuned, and sensitive, to smells and tastes than adults.

Anxiety in patients during the anesthesia procedure can complicate the procedure, as anxiety can manifest physically, causing accelerated pulse, heightened blood pressure, and other symptoms which can increase the risk of undergoing anesthesia, particularly in children.

Prior art attempts to deal with this problem have used scented sprays. These sprays are typically packaged as an aerosol spray, known in the art to be a system for dispersing liquid particles by storing the particles in a pressurized container including a propellant that forces the liquid out of the container when a control is operated, causing the liquid to emerge with velocity as a mist. However, aerosolized scents have numerous problems. First, to use such a product, the mask is inverted and the spray is applied to the inside. However, by the nature of an aerosol, not all of the dispersed liquid will reach the mask. Rather, some amount will be absorbed into the ambient air. This can cause unwanted particular matter in an otherwise sterilized and clean clinical setting.

Also, aerosol dispersants are not ideal for use on a surface. Aerosols are most suitable for use where the propelled liquid is meant to be absorbed into the ambient air (such as an air freshener spray), or wiped into, or off of the surface to which it is applied (such as glass cleaners and insect repellent). However, if aerosolized liquids are sprayed onto a surface and left unattended, tend to accumulate into droplets. If applied to a breathing mask, when the mask is affixed to the patient, this particulate matter on the surface of the mask may drip onto the patient's face, or be inhaled into the mouth of nose. This may be uncomfortable or irritating to the patient, increasing, rather than reducing, the patient's anxiety. Additionally, odorants operate by evaporation and large droplets on the mask may not evaporate rapidly enough to impart any significant odor.

### SUMMARY OF THE INVENTION

The following is a summary of the invention in order to provide a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems in the art, described herein, among other things, is a method of reducing anxiety in a patient comprising: providing a wearable medical device configured for use on a human patient; providing a towelette treated with an odorant; applying the towelette to the wearable medical device such that at least some of the odorant is transferred from the towelette to the wearable medical device; fitting the wearable medical device having the transferred at least some odorant to the human patient; at least some of the transferred at least some odorant vaporizing from the wearable medical device; the human patient inhaling at least some of the vaporized odorant.

In an embodiment of the method, the odorant has a sweet odor.

In a further embodiment of the method, the sweet odor is a fruit odor.

In an embodiment of the method, the wearable medical device comprises an anesthesia breathing mask.

In a further embodiment of the method, the breathing mask is sized and shaped for use with a child.

In a another further embodiment of the method, the applying step comprises wiping down the interior the of side breathing mask.

In an embodiment of the method, the towelette is not treated with an alcohol or anti-microbial agent prior to the applying step.

In an embodiment of the method, before the applying step, the towelette is treated with a one or more agents selected from the group consisting of: a cleaning agent, an anti-bacterial agent, an anti-microbial agent, a dissipant agent, or an evaporant agent.

In an embodiment of the method, the transferred odorant is safely ingestable by a human.

In an embodiment of the method, the odorant comprises from between about 30 to about 60%, by weight or mass, of glycerin.

In an embodiment of the method, the odorant comprises from between about 1% to about 5%, by weight or mass, of carboxymethylcellulose.

In an embodiment of the method, the odorant comprises from between about 1% to about 5%, by weight or mass, of glycol.

In an embodiment of the method, the odorant comprises from between about 0.1% to about 2%, by weight or mass, of sucralose.

### BRIEF DESCRIPTION OF THE DRAWINGS

FIG. 1 depicts an embodiment of a method of reducing anxiety in a patient according to the present disclosure.

FIG. 2 depicts a scented towelette and mask according to the present disclosure.

### DESCRIPTION OF THE PREFERRED EMBODIMENT(S)

The following detailed description and disclosure illustrates by way of example and not by way of limitation. This description will clearly enable one skilled in the art to make and use the disclosed systems and methods, and describes several embodiments, adaptations, variations, alternatives and uses of the disclosed systems and methods. As various changes could be made in the above constructions without departing from the scope of the disclosures, it is intended that all matter contained in the description or shown in the accompanying drawings shall be interpreted as illustrative and not in a limiting sense.

Described herein, among other things, are apparatus and methods for inhibiting the odor of an anesthesia mask, and thereby reducing patient anxiety. The apparatus and methods describes herein generally comprise the use of a towelette treated with a pleasant odorant or flavorant to wipe down a piece of medical equipment, specifically, a piece of non-metallic or plastic equipment, such as a breathing mask, prior to it being positioned on a patient in a location where it can be smelled or tasted.

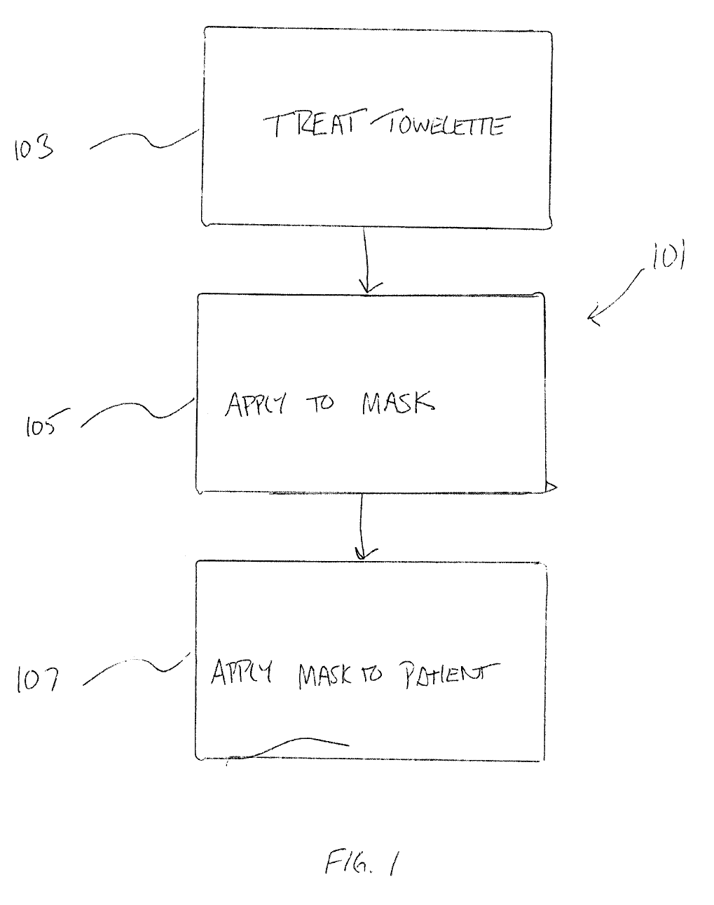

FIG. 1 depicts an embodiment of a method (101) for inhibiting or reducing anxiety in a patient undergoing anesthesia. In the depicted embodiment of FIG. 1, a towelette is treated (103) with an odorant or flavorant. The “towelette” means a typically small and disposable paper or cloth towel. Towelettes are typically individually packaged in airtight and watertight packaging that is yet easy to tear open without the use of cutting tools. In the prior art, such towelettes are typically treated with a cleaning or cleansing agent, such as an alcohol-based anti-microbial compound, and the sealed individual packaging prevents the cleaning agent from evaporating. Thus, the towelette is typically moistened or damp when first removed from the packaging. In the depicted embodiment of FIG. 1, the towelette is treated (103) with an odorant or flavorant. This is preferably in place of a cleaning or cleansing agent, as the towelette will be applied to a new, sterilized, or clean breathing mask, which does not require cleaning or cleansing prior to use. Further, cleaning agents may also impart an unpleasant odor. However, in an alternative embodiment, the towelette may additionally be treated with other agents, such as, but not necessarily limited to, a cleaning or cleansing agent, an anti-bacterial, an anti-microbial, a dissipant, or an evaporant. This step is typically performed at the point of manufacturing, before the towelette is packaged and shipped.

Because the method and apparatus are generally used with children, the preferred odorant or flavorant is typically characterized by sweetness, as there is a memory component to the sense of smell, and a pleasant, sweet smell is more likely to calm a child by rousing fond memories. By way of example and not limitation, a fruit-flavored odorant or flavorant may be used. The precise chemical composition of the odorant or flavorant may vary from embodiment to embodiment, and it its preferred that the odorant or flavorant be suitable for ingestion or inhalation in at least small quantities. Examples include fruit-flavored or fruit-odored oils, such as glycerides. Compounds that generally cause minimal skin irritation and may be safely inhaled or ingested are preferred, to reduce the likelihood of allergicreactions.

When a patient, typically a child, is fitted with a breathing mask, typically but not necessarily as part of an anesthetic procedure, the mask is treated (105) with a towelette immediately before being fitted to the patient. This application (105) comprises wiping down the interior surfaces of the mask with a treated towelette so as to transfer some of the odorant to the interior surface of the mask. The odorant transferred to the interior surface of the mask will then begin to vaporize as dispersants, water, scent, or the oil itself evaporates. This produces a pleasant smell in the vicinity of the mask. The evaporation process may be aided by the flow of oxygen to the mask.

Next, the mask is fitted (107) to the patient. The unpleasant “new plastic” smell of the mask is then covered or suppressed by the stronger and fresher odor of the odorant vapor, because the odorant was recently transferred to the interior of the mask. Further, a medical breathing mask usually covers the mouth and nose and is largely enclosed, having a hose for supplying oxygen and/or other inhalants, and an exhaust hose or port for expelling respiratory byproducts. When the mask is fitted to the patient's face, the concentration of odorant vapor in the largely enclosed compartment defined by the mask interior and the patient's face will generally increase quickly, causing the patient to primarily smell the odorant rather than the mask. The rate of evaporation may be increase by the heat from the patient's respiration into the mask.

Another advantage of the use of towelettes is that the odorant and other compounds with which the towelette is treated can be spread over the mask in a thin film, rather than concentrated in aerosolized droplets. This reduces the waste inherent in aerosol sprays, and avoids the problem of larger droplets accumulating on the mask. Because the towelettes can be wiped across the mask, a thin layer can be applied, which will evaporate at a more desirable rate when fitted to the patient.

The towelette may be treated with a number of chemicals, including, but not necessarily limited to, one or more of the following: glycerin (30-60%), carboxymethylcellulose (1-5%), pentylene glycol (1-5%), sucralose (0.1-2%).

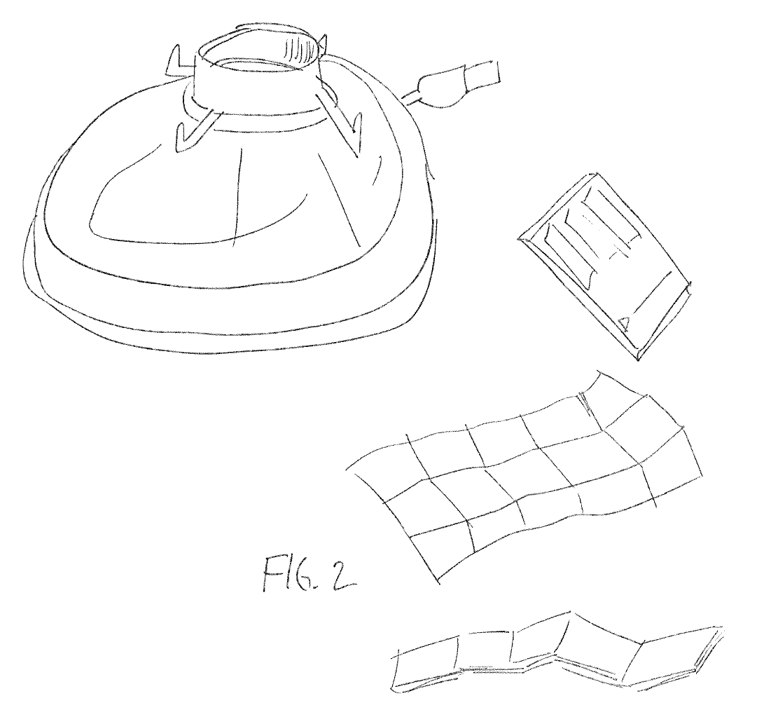

FIG. 2 depicts an embodiment of an anesthesia mask for use in the method according to the present disclosure. FIG. 2 also depicts an embodiment of a towelette for use in the method according to the present disclosure. The depicted towelette is shown individually packaged, partially unfolded, and fully unfolded.

In an embodiment, the odorant or flavorant may be one or more products selected from the group consisting of: Trigg Laboratories product code 8014-01, 21509 (Watermelon), Trigg Laboratories Product code 8012-01, 21501 (Passion Fruit Punch), Trigg Laboratories Product code 8011-02, 21608, 21503 (Kiwi Strawberry), Trigg Laboratories Product code 8018-01, 21501 (Sweet Cherry), or other similar products.

While the invention has been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

1. A method of reducing anxiety in a patient comprising:
providing a wearable medical device configured for use on a human patient;
providing a towelette treated with an odorant;
applying said towelette to said wearable medical device such that at least some of said odorant is transferred from said towelette to said wearable medical device;
fitting said wearable medical device having said transferred at least some odorant to said human patient;
at least some of said transferred at least some odorant vaporizing from said wearable medical device;
said human patient inhaling at least some of said vaporized odorant.

2. The method of claim 1, wherein said odorant has a sweet odor.

3. The method of claim 2, wherein said sweet odor is a fruit odor.

4. The method of claim 1, wherein said wearable medical device comprises an anesthesia breathing mask.

5. The method of claim 4, wherein said breathing mask is sized and shaped for use with a child.

6. The method of claim 4, wherein said applying step comprises wiping down the interior said of side breathing mask.

7. The method of claim 1, wherein said towelette is not treated with an alcohol or anti-microbial agent prior to said applying step.

8. The method of claim 1, wherein, before said applying step, said towelette is treated with a one or more agents selected from the group consisting of: a cleaning agent, an anti-bacterial agent, an anti-microbial agent, a dissipant agent, or an evaporant agent.

9. The method of claim 1, wherein said transferred odorant is safely ingestable by a human.

10. The method of claim 1, wherein said odorant comprises from between about 30 to about 60%, by weight or mass, of glycerin.

11. The method of claim 1, wherein said odorant comprises from between about 1% to about 5%, by weight or mass, of carboxymethylcellulose.

12. The method of claim 1, wherein said odorant comprises from between about 1% to about 5%, by weight or mass, of glycol.

13. The method of claim 1, wherein said odorant comprises from between about 0.1% to about 2%, by weight or mass, of sucralose.
